(19) United States
(12) Patent Application Publication
(10) Pub. No.: US 2012/0259226 A1
(43) Pub. Date: Oct. 11, 2012

(54) ULTRASOUND DIAGNOSTIC APPARATUS

(76) Inventor: Yasufumi TAKAHASHI, Kanagawa (JP)

(21) Appl. No.: 13/441,238

(22) Filed: Apr. 6, 2012

(30) Foreign Application Priority Data
Apr. 8, 2011 (JP) 2011-086346

(51) Int. Cl. A61B 8/14

(52) U.S. Cl. 600/447

(57) ABSTRACT

An ultrasound diagnostic apparatus comprises: an ultrasound probe, the ultrasound probe having a transducer array which transmits ultrasonic waves, receives an ultrasonic echo reflected by a subject, and outputs a reception signal according to the received ultrasonic waves, a signal processor which includes a reception amplifier having an amplifier amplifying the reception signal output from the transducer array and processes the reception signal, a selection unit for switching a current value of a bias current supplied to the amplifier, and a selection switch which performs a switching operation to switch the current value by the selection unit; and a diagnostic apparatus body which produces an ultrasound image according to the reception signal processed by the signal processor of the ultrasound probe.

### BACKGROUND OF THE INVENTION

The present invention relates to an ultrasound diagnostic apparatus, and in particular, to a technique for suppressing the amount of heat generated in an ultrasound probe of an ultrasound diagnostic apparatus, which performs diagnosis on the basis of an ultrasound image produced through transmission and reception of ultrasonic waves from a transducer array in the ultrasound probe.

An ultrasound diagnostic apparatus using an ultrasound image has hitherto been put into practical use in the field of medicine. In general, this type of ultrasound diagnostic apparatus has an ultrasound probe equipped with a transducer array and an apparatus body connected to the ultrasound probe. Ultrasonic waves are transmitted from the ultrasound probe toward a subject, an ultrasonic echo from the subject is received by the ultrasound probe, and the reception signal is electrically processed by the apparatus body to produce an ultrasound image.

In this ultrasound diagnostic apparatus, when ultrasonic waves are transmitted from the transducer array, heat is generated by the transducer array.

Usually, an operator holds an ultrasound probe with one hand and makes a diagnosis while bringing the ultrasound transmission/reception surface of the transducer array into contact with the surface of the subject. Accordingly, the ultrasound probe is accommodated in a small housing of a size such that the operator can easily hold the ultrasound probe with one hand. For this reason, the temperature in the housing of the ultrasound probe increases due to heat generated by the transducer array.

In recent years, an ultrasound diagnostic apparatus is known in which a circuit board for a signal process is embedded in the ultrasound probe, and the reception signal output from the transducer array is subjected to a digital process and transmitted to the apparatus body through wireless communication or wired communication, thereby reducing the effect of noise to obtain a high-quality ultrasound image.

In the ultrasound probe which performs this type of digital process, heat is generated by the circuit board when processing the reception signal, and it is necessary to suppress a rise in temperature in the housing so as to ensure stable operation of each circuit of the circuit board.

As the countermeasure against a rise in temperature of the ultrasound probe, for example, JP 2005-253776 A describes an ultrasound diagnostic apparatus which automatically changes the actuation conditions of the transducer array in accordance with the surface temperature of the ultrasound probe. As the surface temperature increases, the actuation voltage of each transducer of the transducer array, the number of transmission openings, the repetition frequency of transmission pulses, the frame rate, and the like when transmitting ultrasonic waves are reduced, such that the surface temperature of the ultrasound probe is maintained at an appropriate temperature.

JP 2009-148424 A describes an ultrasound diagnostic apparatus which stops the operation of a reception circuit in a probe for a predetermined period, such as a freeze period, a blanking period, a period in which movement of the probe is equal to or smaller than a prescribed value, or a period in which the temperature of the probe is equal to or higher than a prescribed value, thereby suppressing a rise in temperature of the probe due to heat generated by the circuit.

### SUMMARY OF THE INVENTION

In the apparatus described in JP 2005-253776 A which changes the actuation conditions of the transducer array at the time of transmission, it is difficult to handle heat generated at the time of reception in the ultrasound probe which performs the above-described digital process.

In the apparatus described in JP 2009-148424 A which stops the operation of the reception circuit in the probe for a predetermined period, such as a freeze period, a blanking period, a period in which movement of the probe is equal to or smaller than a prescribed value, or a period in which the temperature of the probe is equal to or higher than a prescribed value, while it is possible to reduce heat generated at the time of reception in the ultrasound probe which performs a digital process, the ratio of the predetermined period is generally small compared to the operation time, making it difficult to sufficiently reduce heat generated in the ultrasound probe.

The invention has been accomplished in order to solve the problems in the related art, and an object of the invention is to provide an ultrasound diagnostic apparatus and a method of producing an ultrasound image capable of obtaining a high-quality ultrasound image while suppressing a rise in internal temperature of an ultrasound probe.

In order to solve the above problems, the present invention provides an ultrasound diagnostic apparatus comprising: an ultrasound probe, the ultrasound probe having a transducer array which transmits ultrasonic waves, receives an ultrasonic echo reflected by a subject, and outputs a reception signal according to the received ultrasonic waves, a signal processor which includes a reception amplifier having an amplifier amplifying the reception signal output from the transducer array and processes the reception signal, a selection unit for switching a current value of a bias current supplied to the amplifier, and a selection switch which performs a switching operation to switch the current value by the selection unit; and a diagnostic apparatus body which produces an ultrasound image according to the reception signal processed by the signal processor of the ultrasound probe.

Preferably, the selection unit switches the current value of the bias current supplied to the amplifier between a predetermined first current value and a predetermined second current value greater than the first current value.

Preferably, the signal processor has an analog/digital converter which converts the reception signal amplified by the reception amplifier to a digital signal.

It is preferable that the reception amplifier has a low-noise amplifier, and the selection unit switches a current value of a bias current supplied to the low-noise amplifier.

It is preferable that the reception amplifier has a plurality of amplifiers, and the selection unit switches a current value of a bias current of at least one amplifier.

Preferably, the ultrasound diagnostic apparatus further comprises a temperature measurement unit for measuring temperature in the ultrasound probe, wherein, when the temperature measured by the temperature measurement unit is higher than a predetermined temperature, the selection unit switches the bias current supplied to the amplifier to the first current value.

It is preferable that an elapsed time after the measured temperature measured by the temperature measurement unit exceeds the predetermined temperature is measured, and when the measured temperature is higher than the predetermined temperature after the elapsed time has elapsed a predetermined time, supply of the bias current to the amplifier is stopped.

Preferably, the ultrasound probe transmits the reception signal to the diagnostic apparatus body through wireless communication.

According to the invention, the reception amplifier having the amplifier amplifying the reception signal output from the transducer array and the selection unit switching the current value of the bias current supplied to the amplifier are arranged in the ultrasound probe, and the selection switch performing the switching operation to switch the current value by the selection unit is provided in the ultrasound probe. Therefore, it is possible to obtain a high-quality ultrasound image while suppressing the amount of heat generated in the ultrasound probe.

### BRIEF DESCRIPTION OF THE DRAWINGS

FIG. 1 is a block diagram illustrating the configuration of an ultrasound probe in an ultrasound diagnostic apparatus according to the invention.

FIG. 2 is a block diagram illustrating the configuration of a diagnostic apparatus body in the ultrasound diagnostic apparatus according to the invention.

FIG. 3 is a diagram conceptually illustrating the appearance of the ultrasound probe illustrated in FIG. 1.

FIG. 4 is a graph conceptually illustrating the relation between a bias current and noise of an LNA.

FIG. 5 is a diagram conceptually illustrating an ultrasound image.

### DETAILED DESCRIPTION OF THE INVENTION

Hereinafter, an ultrasound diagnostic apparatus of the invention will be described in detail in connection with a preferred embodiment illustrated in the accompanying drawings.

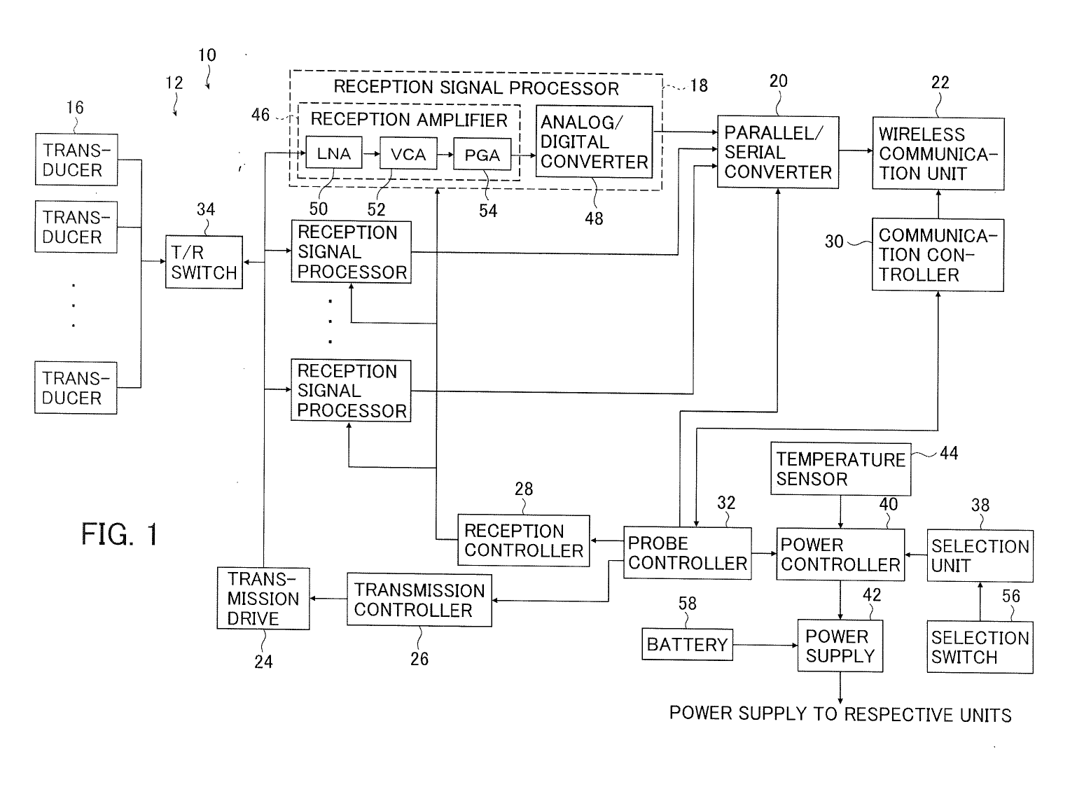

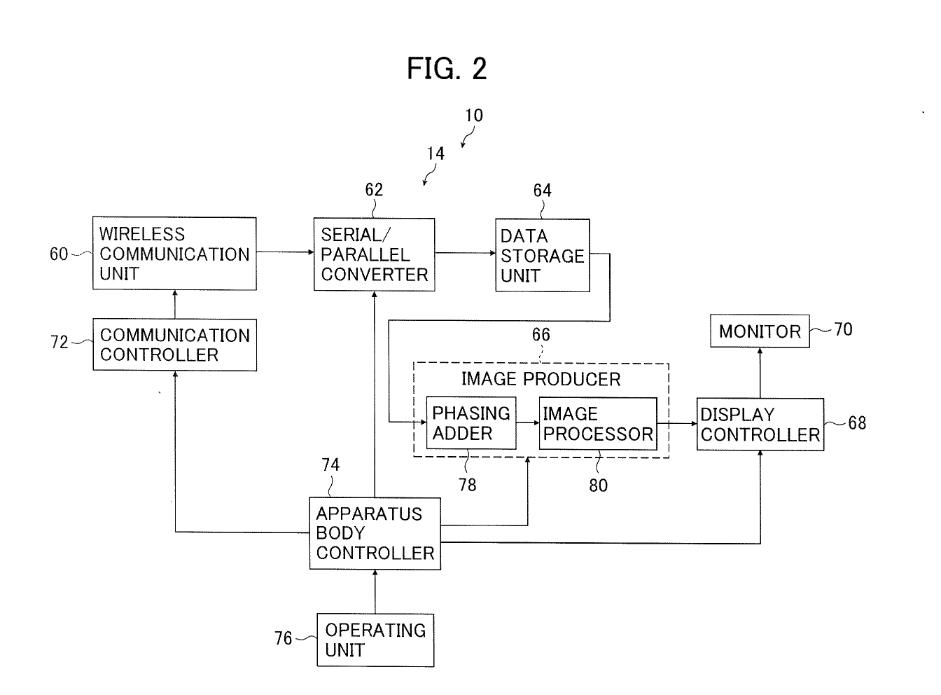

FIG. 1 is a block diagram conceptually illustrating the configuration of an ultrasound probe in an ultrasound diagnostic apparatus of the invention. FIG. 2 is a block diagram conceptually illustrating the configuration of a diagnostic apparatus body in the ultrasound diagnostic apparatus of the invention.

An ultrasound diagnostic apparatus 10 includes an ultrasound probe 12, and a diagnostic apparatus body 14 which is connected to the ultrasound probe 12 through wireless communication.

The ultrasound probe 12 has a plurality of ultrasound transducers 16 which form a plurality of channels of a one or two-dimensional transducer array. Reception signal processors 18 are connected to the respective transducers 16 through a T/R switch 34, and a wireless communication unit 22 is connected to the reception signal processors 18 through a parallel/serial converter 20. A transmission controller 26 is connected to the plurality of transducers 16 through a transmission drive 24. A reception controller 28 is connected to the plurality of reception signal processors 18. A communication controller 30 is connected to the wireless communication unit 22.

Moreover, the ultrasound probe 12 has a power supply 42 which supplies power to the respective units of the ultrasound probe 12. A power controller 40 and a battery 58 are connected to the power supply 42. A temperature sensor 44 and a selection unit 38 are connected to the power controller 40. A selection switch 56 is connected to the selection unit 38.

A probe controller 32 is connected to the parallel/serial converter 20, the transmission controller 26, the reception controller 28, the communication controller 30, and the power controller 40.

The power supply 42 supplies power charged in the battery 58 to the respective units of the ultrasound probe 12, such as the transmission drive 24 and the reception signal processors 18, under the control of the power controller 40.

The power controller 40 performs control such that the power supply 42 supplies a desired amount of power to the respective units of the ultrasound probe 12 in response to an instruction from the probe controller 28.

Moreover, the power controller 40 performs control such that the power supply 42 switches the current value of a bias current supplied to an LNA 50 of the reception signal processor 18 in response to an instruction signal from the selection unit 38.

The selection unit 38 memorizes a first current value and a second current value greater than the first current value as current values of the bias current supplied to the LNA 50 of the reception signal processor 18, and supplies the current value selected in accordance with an operation of the operator using the selection switch 56 to the power controller 40.

In the following description, a mode in which the bias current of the first current value is supplied to the LNA 50 is referred to as a standard image quality mode, and a mode in which the bias current of the second current value is supplied to the LNA 50 is referred to as a high image quality mode.

The switching of the current value of the bias current supplied to the LNA 50 will be described below in detail.

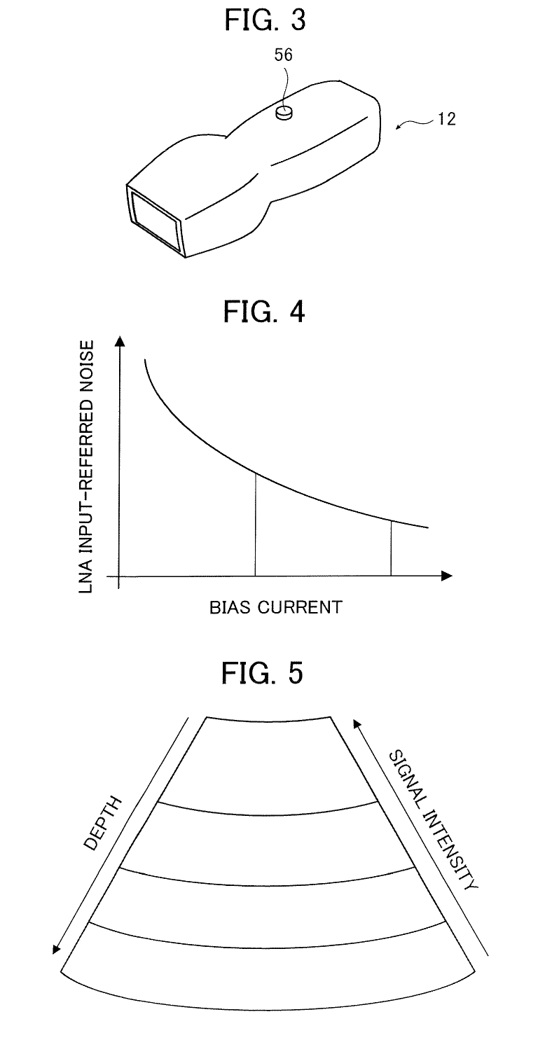

The selection switch 56 is used when the operator performs a selection operation of an image quality mode. As illustrated in FIG. 3, the selection switch 56 is a push button-type switch provided in the housing of the ultrasound probe 12. In this embodiment, in a state where the selection switch 56 is not depressed, the standard image quality mode is selected, and in a state where the selection switch 56 is depressed, the high image quality mode is selected.

The plurality of transducers 16 transmit ultrasonic waves in response to an activation signal supplied from the transmission drive 24, receive an ultrasonic echo from a subject, and output reception signals. Each transducer 16 is constituted by a vibrator in which electrodes are formed at both ends of a piezoelectric body made of piezoelectric ceramic represented by PZT (lead zirconate titanate), a polymer piezoelectric device represented by PVDF (polyvinylidene difluoride), piezoelectric single crystal represented by PMN-PT (lead magnesium niobate-lead titanate solid solution), or the like.

If a pulsed or continuous-wave voltage is applied across the electrodes of the vibrator, the piezoelectric body expands and contracts, and pulsed or continuous-wave ultrasonic waves are produced from the respective vibrators and synthesized to form an ultrasonic beam. When receiving the propagating ultrasonic waves, the respective vibrators expand and contract to produce electric signals, and the electric signals are output as the reception signals of the ultrasonic waves.

The T/R switch 34 selects M ultrasound transducers from among N ultrasound transducers, and respectively connects the selected M ultrasound transducers to M transmission and reception circuits.

The transmission drive 24 includes, for example, a plurality of pulsers, adjusts the delay amount of the activation signal on the basis of a transmission delay pattern selected by the transmission controller 26 such that the ultrasonic waves transmitted from the plurality of transducers 16 form an ultrasonic beam having a width enough to cover an area of a tissue in the subject and supplies the activation signal to the plurality of transducers 16 through the T/R switch 34.

The reception signal processor 18 of each channel processes the reception signal output from the corresponding transducer 16 to produce sample data including area information of the tissue under the reception controller 28.

The reception signal processor 18 has a reception amplifier 46 and an analog/digital converter 48.

The reception amplifier 46 amplifies the reception signal output from the transducer 16.

The reception amplifier 46 has an LNA (Low Noise Amplifier) 50, a VCA (Voltage Controlled Attenuator) 52, and a PGA (Programmable Gain Amplifier) 54.

The LNA 50 is supplied with the bias current from the power supply 42 to amplify the reception signal output from the transducer 16.

As described above, in the invention, the current value of the bias current supplied to the LNA 50 is switched between a predetermined first current value (standard image quality mode) and a second current value (high image quality mode) greater than the first current value by the selection means 38 in accordance with an operation of the selection switch 56.

FIG. 4 schematically illustrates the relation between the bias current supplied to the LNA 50 and input-referred noise. As illustrated in FIG. 4, the larger the current value of the bias current to be supplied, the smaller input-referred noise of the LNA. That is, when the bias current of the first current value is supplied to the LNA 50, the S/N ratio of the LNA 50 decreases, and when the bias current of the second current value is supplied to the LNA 50, the S/N ratio of the LNA 50 increases.

When the bias current of the first current value is supplied to the LNA 50 (standard image quality mode), the current value of the bias current is small. Accordingly, while noise increases and image quality is degraded in the ultrasound image produced from the reception signal amplified by the LNA 50 due to decrease of the S/N ratio, it is possible to reduce heat generated in the LNA 50, that is, heat generated in the ultrasound probe 12.

On the other hand, when the bias current of the second current value is supplied to the LNA 50 (high image quality mode), the current value of the bias current is large. Accordingly, while heat generated in the LNA 50 (ultrasound probe 12) increases, the S/N ratio increases. Thus, in the ultrasound image produced from the reception signal amplified by the LNA 50, noise decreases and image quality is improved. FIG. 5 is a diagram conceptually illustrating an ultrasound image.

As illustrated in FIG. 5, in the ultrasound image, the larger the distance (depth) from the ultrasound probe in the depth direction, the smaller the reception signal of the ultrasonic echo reflected by the subject. For this reason, during imaging in the standard image quality mode, in a deep region, the ratio of noise relative to the reception signal increases, and image quality (resolution) is deteriorated. Meanwhile, in a shallow region, the reception signal is large. For this reason, in the standard image quality mode, the ratio of noise relative to the reception signal decreases, such that degradation of image quality is small.

Accordingly, the operator explores the subject in the standard image quality mode in which a small amount of heat is generated to find a desired region of interest, and then depresses the selection switch 56 to switch the mode to the high image quality mode, thereby obtaining a high-quality ultrasound image capable of being submitted for close inspection.

As described above, when a configuration is made in which the operation of the reception circuit in the probe is stopped for a predetermined period, such as a freeze period, a blanking period, a period in which movement of the probe is equal to or smaller than a prescribed value, or a period in which the temperature of the probe is equal to or higher than a prescribed value, the ratio of the predetermined period is very small compared to the operation time, making it difficult to sufficiently reduce heat generated in the ultrasound probe.

In contrast, the invention has a configuration in which the current value of the bias current supplied to the LNA 50 is switched by the selection unit 38 between the predetermined first current value and the second current value greater than the first current value in accordance with an operation of the selection switch 56. For this reason, it is possible to switch the mode between the standard image quality mode in which a small amount of heat is generated and the high image quality mode in which close inspection can be performed at the timing desired by the operator. Therefore, in the ultrasound probe which performs a digital process, it is possible to suppress the amount of heat generated from the circuits in the probe and to obtain a high-quality ultrasound image.

The LNA 50 supplies the amplified reception signal to the VCA 52.

The VCA 52 attenuates the reception signal supplied from the LNA 50 in accordance with the depth of the reception signal. The VCA 52 supplies the attenuated reception signal to the PGA 54.

The PGA 54 amplifies the reception signal supplied from the VCA 52 and supplies the amplified reception signal to the analog/digital converter 48.

The analog/digital converter 48 samples the analog reception signal supplied from the PGA 54 to produce digital sample data. The analog/digital converter 48 supplies the digital sample data to the parallel/serial converter 20.

The parallel/serial converter 20 converts the parallel sample data produced by the reception signal processors 18 of the multiple channels to serial sample data.

The wireless communication unit 22 modulates carriers on the basis of the serial sample data to produce a transmission signal, supplies the transmission signal to an antenna, and transmits radio waves from the antenna to transmit the serial sample data. As the modulation system, for example, ASK (Amplitude Shift Keying), PSK (Phase Shift Keying), QPSK (Quadrature Phase Shift Keying), 16QAM (16 Quadrature Amplitude Modulation), or the like is used.

The wireless communication unit 22 performs wireless communication with the diagnostic apparatus body 14 to transmit the sample data to the diagnostic apparatus body 14 and to receive various control signals from the diagnostic apparatus body 14, and outputs the received control signals to the communication controller 30. The communication controller 30 performs control such that the wireless communication unit 22 transmits the sample data with a transmission radio-field intensity set by the probe controller 32, and outputs various control signals received by the wireless communication unit 22 to the probe controller 32.

The probe controller 32 controls the respective units of the ultrasound probe 12 on the basis of various control signals transmitted from the diagnostic apparatus body 14.

The ultrasound probe 12 may be an external probe, such as a linear scan type, a convex scan type, or a sector scan type, or may be a probe for an ultrasound endoscope, such as a radial scan type.

In this embodiment, it is preferable that the temperature sensor 44 is provided to measure the temperature in the ultrasound probe 12.

The temperature sensor 44 supplies the measured temperature to the power controller 40. When the value of the supplied temperature is equal to or higher than a predetermined temperature, the power controller 40 performs control such that the power supply 42 sets the bias current supplied to the LNA 50 to the first current value regardless of the presence/absence of the operation of the selection switch 56. Therefore, it is possible to prevent an excessive rise in temperature of the ultrasound probe 12.

Further, an elapsed time after the value of the temperature measured by the temperature sensor 44 is equal to or higher than the predetermined temperature and the bias current supplied to the LNA 50 is set to the first current value is measured, and even if a given time has elapsed, when the measured temperature of the temperature sensor 44 is equal to or higher than the predetermined temperature, the supply of the bias current to the LNA 50 may be stopped.

It is preferable that the temperature sensor 44 continues to measure the temperature even after the supply of the bias current to the LNA 50 has been stopped and resumes the supply of the bias current to the LNA 50 when the temperature is lower than the predetermined temperature. Alternatively, a monitor 70 may perform display indicating usability. Therefore, it is possible to prevent an excessive rise in temperature of the ultrasound probe 12.

The diagnostic apparatus body 14 has a wireless communication unit 60. A data storage unit 64 is connected to the wireless communication unit 60 through a serial/parallel converter 62, and an image producer 66 is connected to the data storage unit 64. The monitor 70 is connected to the image producer 66 through a display controller 68.

A communication controller 72 is connected to the wireless communication unit 60, and an apparatus body controller 74 is connected to the serial/parallel converter 62, the image producer 66, the display controller 68, and the communication controller 72. An operating unit 76 which is used when the operator performs an input operation is connected to the apparatus body controller 74.

The operating unit 76 is used to set an imaging menu, imaging conditions, and the like, and to perform an input operation to instruct imaging of the subject. The operating unit 76 may include a keyboard, a mouse, a trackball, a touch panel, and the like which are used when the operator performs the input operation.

The wireless communication unit 60 performs wireless communication with the ultrasound probe 12, and transmits various control signals to the ultrasound probe 12. Besides, the wireless communication unit 60 demodulates a signal received by the antenna to output serial sample data.

The communication controller 72 performs control such that the wireless communication unit 60 transmits various control signals with a transmission radio-field intensity set by the apparatus body controller 74.

The serial/parallel converter 62 converts the serial sample data output from the wireless communication unit 60 to parallel sample data. The data storage unit 64 includes a memory, a hard disk, or the like, and stores the sample data for at least one frame converted by the serial/parallel converter 62.

The image producer 66 performs a reception focus process on the sample data for every frame read from the data storage unit 64 to produce an image signal representing an ultrasound diagnostic image. The image producer 66 includes a phasing adder 78 and an image processor 80.

The phasing adder 78 performs the reception focus process by selecting one reception delay pattern from among a plurality of reception delay patterns stored in advance in accordance with the reception direction set in the apparatus body controller 74, giving the delay to each of a plurality of complex baseband signals represented by the sample data on the basis of the selected reception delay pattern, and adding the complex baseband signals. With this reception focus process, the focus of the ultrasonic echo is narrowed to produce a baseband signal (sound ray signal).

The image processor 80 produces a B-mode image signal, which is tomographic image information relating to the tissue of the subject, on the basis of the sound ray signal produced by the phasing adder 78. The image processor 80 includes an STC (Sensitivity Time Control) unit, an interpolator, and a DSC (Digital Scan Converter). The STC unit corrects attenuation depending on the distance in accordance with the depth of the reflection position of the ultrasonic wave for the sound ray signal. The interpolator performs an interpolation process on a missing frame of the sound ray signal by intermittent transmission and reception of ultrasonic waves in a temperature rise suppression mode described below. The DSC converts (raster-converts) the sound ray signal corrected by the STC unit to an image signal based on a normal television signal scan system, and performs a necessary image process, such as a gradation process, to produce a B-mode image signal.

The display controller 68 displays an ultrasound diagnostic image on the monitor 70 on the basis of the image signal produced by the image producer 66. The monitor 70 includes, for example, a display, such as an LCD, and displays the ultrasound diagnostic image under the control of the display controller 68.

The apparatus body controller 74 controls the respective units of the ultrasound diagnostic apparatus 10 in accordance with an operation of the operator using the operating unit 76.

Although in the diagnostic apparatus body 14, the serial/parallel converter 62, the image producer 66, the display controller 68, the communication controller 72, and the apparatus body controller 74 are constituted by a CPU and an operation program which causes the CPU to perform various processes, these may be constituted by digital circuits.

Next, the operation of the ultrasound diagnostic apparatus 10 will be described.

If the operator brings the ultrasound probe 12 into contact with the surface of the subject and starts imaging, the transmission controller 26 controls the transmission drive 24 on the basis of the control signals from the apparatus body controller 74. The transmission drive 24 drives the transducers 16 on the basis of the control signals, an ultrasonic beam is transmitted from each transducer 16, and each transducer 16 receives an ultrasonic echo from the subject and outputs a reception signal.

The reception signal output from each transducer 16 having received the ultrasonic echo from the subject is supplied to the corresponding reception signal processor 18. The reception signal supplied to the reception signal processor 18 is sequentially converted to sample data. The sample data is converted to serial sample data by the parallel/serial converter 20, and the serial sample data is transmitted from the wireless communication unit 22 to the diagnostic apparatus body 14 in a wireless manner. The sample data received by the wireless communication unit 60 of the diagnostic apparatus body 14 is converted to parallel data by the serial/parallel converter 62, and the parallel data is stored in the data storage unit 64. The sample data for every frame is read from the data storage unit 64, an image signal is produced by the image producer 66, and an ultrasound diagnostic image is displayed on the monitor 70 on the basis of the image signal by the display controller 68.

The invention has a configuration in which the selection unit 38 switches the current value of the bias current supplied to the LNA 50 between the first current value and the second current value greater than the first current value in accordance with an operation of the selection switch 56 by the operator.

As described above, the current value of the bias current supplied to the LNA 50 is switched between the first current value and the second current value greater than the first current value, thereby switching between the standard image quality mode in which a small amount of heat is generated and the high image quality mode in which close inspection can be performed. Therefore, in the ultrasound probe which performs a digital process, it is possible to suppress the amount of heat generated from the circuits in the probe and also to obtain a high-quality ultrasound image.

The invention is basically as described above.

Although the invention has been described in detail, the invention is not limited to the foregoing embodiment, and various improvements or modifications may be made within the scope without departing from the gist of the invention.

For example, although the ultrasound diagnostic apparatus in the illustrated example has a configuration in which the reception amplifier 46 amplifying the reception signals from the transducers has the LNA 50, the VCA 52, and the PGA 54, the invention is not limited thereto. The reception amplifier 46 may have other amplifiers or a plurality of amplifiers.

Although in the illustrated example, a configuration in which the bias current of the LNA 50 is switched has been described, the invention is not limited thereto. A configuration in which the bias current of the PGA 54 is switched or a configuration in which the bias current of each of the LNA 50 and the PGA 54 is switched may be made. When there are a plurality of amplifiers which amplify the reception signals from the transducers, a configuration in which the bias current of at least one of the amplifiers is switched may be made.

Although in the illustrated example, the bias current of the LNA 50 is switched in two stages, the invention is not limited thereto. A configuration in which the bias current of the LNA 50 is switched in three stages or more may be made.

Although in the illustrated example, the ultrasound probe 12 and the diagnostic apparatus body 14 perform signal transmission and reception through wireless communication, the invention is not limited thereto. A configuration in which the signal transmission and reception are performed by wired communication means may be made.

What is claimed is:

1. An ultrasound diagnostic apparatus comprising:
an ultrasound probe, the ultrasound probe having a transducer array which transmits ultrasonic waves, receives an ultrasonic echo reflected by a subject, and outputs a reception signal according to the received ultrasonic waves, a signal processor which includes a reception amplifier having an amplifier amplifying the reception signal output from the transducer array and processes the reception signal, a selection unit for switching a current value of a bias current supplied to the amplifier, and a selection switch which performs a switching operation to switch the current value by the selection unit; and
a diagnostic apparatus body which produces an ultrasound image according to the reception signal processed by the signal processor of the ultrasound probe.

2. The ultrasound diagnostic apparatus according to claim 1,
wherein the selection unit switches the current value of the bias current supplied to the amplifier between a predetermined first current value and a predetermined second current value greater than the first current value.

3. The ultrasound diagnostic apparatus according to claim 1,
wherein the signal processor has an analog/digital converter which converts the reception signal amplified by the reception amplifier to a digital signal.

4. The ultrasound diagnostic apparatus according to claim 1,
wherein the reception amplifier has a low-noise amplifier, and the selection unit switches a current value of a bias current supplied to the low-noise amplifier.

5. The ultrasound diagnostic apparatus according to claim 1,
wherein the reception amplifier has a plurality of amplifiers, and the selection unit switches a current value of a bias current of at least one amplifier.

6. The ultrasound diagnostic apparatus according to claim 2, further comprising:
a temperature measurement unit for measuring temperature in the ultrasound probe,
wherein, when the temperature measured by the temperature measurement unit is higher than a predetermined temperature, the selection unit switches the bias current supplied to the amplifier to the first current value.

7. The ultrasound diagnostic apparatus according to claim 6,
wherein an elapsed time after the measured temperature measured by the temperature measurement unit exceeds the predetermined temperature is measured, and when the measured temperature is higher than the predetermined temperature after the elapsed time has elapsed a predetermined time, supply of the bias current to the amplifier is stopped.

8. The ultrasound diagnostic apparatus according to claim 1,
wherein the ultrasound probe transmits the reception signal to the diagnostic apparatus body through wireless communication.
